(19) United States
(12) Patent Application Publication
(10) Pub. No.: US 2014/0350491 A1
(43) Pub. Date: Nov. 27, 2014

(54) Toe JEMs: A decorative, callous (corn) cover for toes compromised of rhinestones, gems, or other similar material.

(71) Applicant: Angela Lynnette Bryant-Brown, Chantilly, VA (US)

(72) Inventor: Angela Lynnette Bryant-Brown, Chantilly, VA (US)

(21) Appl. No.: 14/286,867

(22) Filed: May 23, 2014

Related U.S. Application Data
(60) Provisional application No. 61/826,756, filed on May 23, 2013.

(51) Int. Cl. A61F 13/06; A41D 27/08

(52) U.S. Cl. CPC A61F 13/068; A41D 27/08; A61F 13/063; A61F 2013/00285
USPC 604/293; 2/244

(57) ABSTRACT

Toe JEMs is a decorative callous (corn) cover for toes compromised with rhinestones, gems, or other similar material and applied with a non-toxic skin adhesive. My invention addresses the issue of the unattractive appearance of a toe callous (corn) many women struggle with today. It gives women and or men a cosmetic option for their blemished toes. Instead of hiding their feet during the warm seasons of the year, Toe JEMs gives people the confidence to wear thong sandals, flip-flops, and open toe shoes or to just walk barefooted in public. Toe JEMs are a cosmetic answer to toe jams.

### CROSS REFERENCE

Provisional Application No.—61/826,756 and Attorney Docket No. 506413350

### BACKGROUND OF THE INVENTION

### Problem Solved

Millions of women, and even some men, ashamedly cover their feet during the warm season of the year due to unattractive calluses (corns) on their toes. Even when treated or removed, the skin discoloration (mainly in women of color) doesn't come back leaving an unsightly blemish.

Toe JEMs are an inexpensive solution for a person's Toe JAMs.

There is no other invention like mine since toe rings and toenail designs don't address the toe corn (callous) issue. My invention, Toe JEMs addresses the toe corn (callous) issue many women have previously struggled with and continue to even today.

### DETAILED DESCRIPTION OF THE INVENTION

As stated above, millions of women, and even some men, ashamedly cover their feet during the warm seasons of the year due to the unattractive calluses (corns) on their toes. Even when treated or removed, the skin discoloration (mainly in women of color) doesn't come back leaving an unsightly blemish. The invention of Toe JEMs claimed here offers a suitable solution to this problem.

This decorative toe corn (callous) cover (Toe JEMs) not only covers the affected area but beautifies it, giving women the confidence to wear open toe shoes, go swimming without swim shoes or just walk barefooted around others.

The decorative corn cover, Toe JEMs, invention differs from what currently exists on the market. To my knowledge there is nothing like it being offered on the market. There are toe rings and toenail designs but nothing to camouflage corns (calluses) on toes.

In addition, when increased in size, the decorative corn covers could be applied to any area of the body as a gem tattoo-like display. For example, it could be used to camouflage stretch marks around the navel.

### The Version of The Invention Discussed Here Includes

1. Finger/toenail size rhinestones and/or other similar materials

2. A non-toxic, clear liquid that dries to a laminate-like lacquer finish

3. A non-toxic, water resistant skin adhesive

4. A small, sponge top applicator for skin adhesive

5. Heavy wax paper and plastic wrap for packaging

### Relationship Between The Components

The (1) rhinestone design (Toe JEMs) would be set in (2) a non-toxic, clear liquid solution so that when it dries it looks like a very thin, round, laminate-like covering. The (3) non-toxic, water resistant adhesive will be applied on the backside of the (2) decorative corn cover (Toe JEMs) and (4) directly to the callus/skin. The decorative corn cover will be placed, adhesive side down, on (5) wax paper before placed in a plastic packaging for distribution.

### How The Invention Works

The (1) decorative callus/corn cover (Toe JEMs) will be set in (2) a non-toxic, clear liquid that when it dries it looks like a very thin, round shaped laminate-like gem covering. The (3) non-toxic, water resistant adhesive will be pre-applied on the back side of the (2) round shaped decorative callus/corn cover (Toe JEMs) and then applied directly to the callus/corn. After selecting the needed size corn cover from a variety of sizes provided in the package on (5) heavy wax paper, the (2) nontoxic adhesive decorative callus/corn cover (Toe JEMs) will be placed adhesive side down on the lightly adhesive coated corn/callous (4) and pressed gently but firmly in place for 30-45 seconds until set.

### How To Make The Invention

On a non-stick or plastic mold device comprised of mini circular but flat bottom pockets ranging from ⅛ to ½ inch in diameter, drops of the non-toxic, clear liquid solution will be placed in each pocket and then using a pointy stick-like instrument the rhinestones or other similar decorative materials will be strategically placed in the non-toxic, clear liquid to create the desired design before it dries completely creating a flexible, laminate-like round rhinestone callus/corn cover (Toe JEMs). Once completely dry, the decorative callus/corn cover (Toe JEMs) is removed from its pocket and a nontoxic, water resistant skin adhesive is applied to the backside of the decorative callous/corn cover, then placed on a precut wax paper before its placed in its packaging.

For the invention and creation of Toe JEMs all of the above mentioned elements are necessary.

Another Toe JEMs application option is to apply a callus/corn remover medication to the backside of it, which would make the invention dual in purpose, treating the corn while allowing the toe to be exposed in a more attractive way.

Yet another application of the Toe JEMs decorative callus/corn cover would be to bypass the clear liquid solution and just create decorative callus/corn designs on a tacky/sticky sheet of paper with the bottom of the design facing up. After applying a light coat of water resistant, nontoxic adhesive to the surface of the callus/corn area, press the decorative callus/corn cover onto it, holding in place for 30-45 seconds before removing the tacky paper leaving the decorative callus/corn cover on the corn/callous.

### How To Use The Invention

The person using the decorative callus/corn covers (Toe JEMs) would simply remove the desired size from the wax paper and after applying additional nontoxic, water resistant adhesive via the small, sponge top plastic applicator, if desired, they would place the decorative callus/corn cover (Toe JEMs) on the callous/corn and firmly but gently press for 30-45 seconds. Once set in place, the person is free to wear flip-flops, thong sandals, and other open toe shoes they wouldn't normally be comfortable wearing. With decorative callus/corn covers (Toe JEMs), women and or men can walk in confidence year round. Note: For comfort reasons, Toe JEMs designed using rhinestones or other similar material should not be worn with tight, closed toe shoes.

Additionally, this invention could be designed to incorporate callus/corn removal medication to the adhesive making it a more attractive option than what is currently on the market (i.e. Dr. Scholl's) while removing the undesirable callus/corn.

Also, when increased in size, the decorative callus/corn covers could be applied to any area of the body as a gem tattoo-like display. For example, it could be used to camouflage stretch marks, unsightly skin blemishes and scars or just worn for fun.

### BRIEF DESCRIPTION OF DRAWINGS

FIG. 1 is a top view of the Toe JEM applied to the toe (callus/corn).

FIG. 2 is the actual Toe JEM. The broken line circle surrounding the rhinestone flower design represents the transparent laminate-like substance embedding the rhinestone flower design.

FIG. 3 is a side view of FIG. 2.

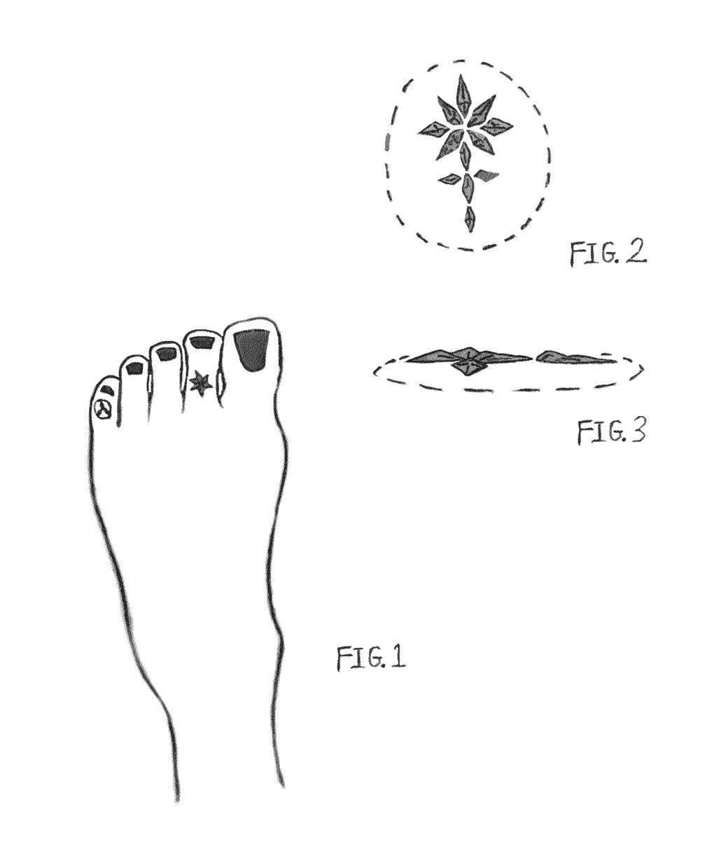

I claim:

1. Toe JEMs comprising: Finger/toenail size rhinestone gems and/or other similar materials, a non-toxic, clear liquid that dries to a laminate-like lacquer finish, a nontoxic, skin adhesive, a small, sponge top applicator of non-toxic, water resistant skin adhesive, wherein rhinestone or other decorative material is used to design a callous cover for a toe by setting the corn cover design in the non-toxic, clear liquid solution so that when it dries it looks like a very thin, round, laminate-like covering, which would then have a non-toxic, water resistant skin adhesive applied to the backside of the decorative callus cover and or the surface of the callus directly; Then the nontoxic adhesive decorative corn cover would be pressed, adhesive side down, directly onto the toe callous, thus covering the callus in part or whole.

2. Toe JEMs of claim 1 further compromising: instead of embedding the rhinestone design in the clear liquid, create the rhinestone design on a tacky or sticky sheet of paper with the bottom of the rhinestones facing up; after applying a light coat of adhesive to the surface of the corn area, press the rhinestone corn cover design onto it and hold for 30-45 seconds before removing the tacky paper, leaving the Toe JEM on the callous.

3. Toe JEMs of claim 1 further compromising can also be used as a decorative, medicated callous removal pad, by applying an adhesive callus remover medication to the underside of the Toe JEM, treating the callus while allowing the toe to be exposed in a more attractive way.

4. Toe JEMs of claims 2 further compromising: when designed in various larger sizes, can be applied to any area of the body as a decorative tattoo-like display.
